(19) United States
(12) Patent Application Publication
(10) Pub. No.: US 2004/0022860 A1
(43) Pub. Date: Feb. 5, 2004

(54) Release composition and method of preparation

(76) Inventors: Von Walter Johson, Christchurch (NZ); Joan Frances Pearson, Christchurch (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 10/362,464

(22) PCT Filed: Aug. 22, 2001
(86) PCT No.: PCT/NZ01/00168

(30) Foreign Application Priority Data
Aug. 22, 2000 (NZ) 506487
Aug. 22, 2000 (NZ) 506488

(51) Int. Cl. A61K 39/00; A61K 9/14; A61K 45/00

(52) U.S. Cl. 424/488; 424/93.7; 424/184.1

(57) ABSTRACT

A method of producing a thermo-stable biodegradable thermo-stable biomatrix for storage of biological materials in disclosed. The biomatrix contains a bio-polymer selected from the group of xanthan gum, acacia gum, guar gum, gellan, starch or a combination therof. The biological material can be a wide range of materials including; a bio-inoculant such as Rhizobium, or any microorganism or cellular organism. The biomatrix can be formulated for fast or slow release of the biological materials and maintains activity of the biological materials after being stored for months. In use the biomatrix can be applied in the form of a pellet or mixed with liquid and applied to seeds, plants or soil.

### TECHNICAL FIELD

The present invention relates to a release composition and method of preparation. More specifically the present invention relates to a composition preferably of a type that allows the release of a compound of biological materials contained within the composition once the composition is placed in water or other solvent.

### BACKGROUND ART

For the purpose of this specification the term “biological material” is used to encompass, but is not limited to, any or all of the following: a bio-inoculant, a micro-organism, biological cells, a part or parts of biological cells, pharmaceuticals, enzymes, hormones, proteins and other biochemicals, unstable compounds and compositions (both biological and non-biological); and a combination of these.

Two known problems are associated with the industrial or agricultural application of biological materials. Firstly, there is the difficulty of maintaining the biological materials in a viable state until they are used or required, and especially during the period in which they may be incorporated in a release mechanism for delivery of the biological material.

Secondly, there may be a need for further storage before the release of the biological material, once it has been applied or distributed to the intended substrate. The delay of release can be for a number of reasons. For example: the composition may need to be stored after manufacture so as to be ready when needed (convenience factor); there may need to be a time delay to the point at which release of the biological material starts, so that the material is released at the intended time for the intended purpose.

In this respect, an example is the release of a hormone for spring growth, intended for consumption by young steers or calves when they start grazing. The delay can also be intended to engender a weedicide, herbicide or insecticide with long-term action after the immediate release into the substrate.

For the purposes of this specification the term “substrate” is used to encompass, but is not limited to, agricultural, horticultural, forestry or other commercial substrates, such as grasses and crops, soils (etc); water, waste water, skins of animals and tissues of animals; and solids such as sands and gravels.

There have been numerous solutions disclosed for encapsulating products for fast release of compounds to substrates.

An example of such a granule can be found in U.S. Pat. No. 6,087,306. This patent discloses a granule, suitable for aqueous spray application, after mixing of the granules with water in a tank, and application through a standard sprayer. The granule incorporates a wetable, dispersible, or water-soluble granule, with an agrochemical or other ingredient suitable for application by aqueous spray. The non-ionic, or predominately non-ionic, water-swellable gums and polymers are disclosed as including microbial polysaccharides such as dextran, gellan gum, and xanthan gum, along with other polysaccharides. These gums and the active ingredient are combined along with an inert filler and extruded as a paste. This paste is dried and the result formed into granules in an extruder. The inert material is preferably water-swellable. The active ingredient is disclosed as a herbicide, insecticide, nematocide, fungicide or plant growth regulator. A wetter or other adjuvant is optionally added.

U.S. Pat. No. 4,859,377 discloses the use of starch to encapsulate a release granulation. The starch is used substantially as an agent without cross-linking. This also discloses, however, that the starch needs to be treated in order to ensure that it is insect digestible, such that the biological agent being carried within the starch capsule reaches the desired destination.

U.S. Pat. No. 4,563,344 discloses elements for both quick and slow release pesticides. The biological chemical is enclosed in a capsule incorporating peat, fine and coarse chaff and the woody portions of corncobs. The combinations and proportions are combined in a pre-determined manner which affect the rate of release.

U.S. Pat. No. 4,434,231 discloses preparation of pellets which incorporate a bio-matrix gel, by the addition of crushed silica which is then homogenised. The resulting pellets are 3 mm in diameter, 33 mm in length and cylindrical in shape. However, there is no disclosure of whether the resultant pellets are fast acting or slow acting or how they breakdown or are activated to break down.

However, all the above disclosed methods of conveying a biological agent to the soil or to a substrate (as herein before defined) disclose disadvantages: the matrix incorporating a biological material needs to be especially treated (as in U.S. Pat. No. 4,859,377) in order to be adapted to break down at the desired end point and time (for example, inside an insect). Certain types of encapsulating material will only incorporate chemicals, not all types of biological materials (U.S. Pat. No. 4,563,344). Some of the compositions are disclosed as being most appropriately applied in an aqueous spray for chemical insecticides like Lambd-eyhalodhrin and Pinmicarb and thus may not be useful for biological materials. (U.S. Pat. No. 6,087,306). Thus many more steps are required to get the biological material to the intended substrate. This in turn increases the inconvenience and cost of application of the biological material.

It is an object of the present invention to provide a composition and method of preparation, which method is both quick and simple, to provide a thermo-stable bio-degradable medium for transfer of a biological material to a substrate, at which point it breaks down quickly on contact with water.

It is a further object of the present invention to address the foregoing problems or at least to provide the public with a useful choice.

Further aspects and advantages of the present invention will become apparent from the ensuing description which is given by way of example only.

### DISCLOSURE OF INVENTION

### DISCLOSURE OF INVENTION

According to one aspect of the present invention there is provided a method of preparation of a release composition, which method comprises the steps of:

(a) preparing a biodegradable bio-matrix either as a gel or a liquid, incorporating a biological material;

(b) preparing a dry powder of one or more inert compounds; selected from the group: talc, bentonite; diatomaceous ea; and a combination thereof; and

(c) mixing the preparation of step (a) and the preparation of step (b) to form a homogenous mix;

and wherein the bio-matrix is selected from the group: xantan gum; acacia gum; guar gum; gellan; starch; and a combination thereof;

and wherein the biological material is selected from: a bio-inoculant, a micro-organism, biological cells, a part or parts of a biological cell, a vaccine, at least one pharmaceutical compound, at least one enzyme, at least one hormone, at least one protein; at least one bio-chemical compound, at least one biologically unstable composition; and a combination thereof;

and wherein said composition is thermo-stable and bio-stable when stored with no more than trace amounts of water.

According to another further aspect of the present invention there is provided a process for the preparation of a release composition as described above, wherein said biological material is combined with a chemical composition.

Advantageously, said organic material may be selected from agricultural materials or waste agricultural materials, for example: corn cobs; chaff, straw. Advantageously, said inert compounds may include peat and sands.

According to another further aspect of the present invention there is provided a process for the preparation of a release composition as described above wherein said method includes a further step after step (c) as follows:

(d) mixing the composition formed in step (c) with at least one clay in powdered form.

According to another further aspect of the present invention there is provided a process for the preparation of a release composition as described above wherein the inert compound used in step (b) and step (d), if present, is clay which is selected from: diatomaceous earth, talc, bentonite, a combination thereof.

According to a further aspect of the present invention there is provided the process for the preparation of a release composition as described above wherein the clay in step (d) (if present) is powdered bentonite.

According to another further aspect of the present invention there is provided a process for the preparation of a release composition as described above, wherein said method includes a further step, after step (c) as follows:

(e) repeating steps (a) to (c) one or more times with a different bio-matrix and/or a different biological material; and mixing the homogenous mix resultant from each step (c).

According to another further aspect of the present invention there is provided a process for the preparation of a release composition as described above, wherein said method includes a further step after step (d) as follows:

(f) mixing the resultant mix of step (d) with water, to form a dough.

According to another further aspect of the present invention there is provided a process for the preparation of a release composition as described above, wherein said method includes a further step after step (f) as follows:

(g) forcing through or extruding the dough of step (f) in a pelletiser or granulator to form pellets;

(h) drying the pellets or granules at room temperature.

The pellets or granules may have a size of 0.1-20 mm diameter. The pellets may be up to 30 mm long.

Advantageously, the pellets resulting from step (g) may be in the range 0.1 mm to 10 mm in diameter, and up to 10 mm in length.

According to a further aspect of the present invention there is provided the method of preparation of a slow release or delayed release composition substantially as described above wherein said bio-degradable bio-matrix has between 15 to 40% water by weight at the end of step (g).

In the Applicant's experience, a slow release composition is bio-stable in that until the composition comes into contact with substantial amounts of water, it is both bio-stable and thermo-stable. However, once water is introduced the matrix breaks down slowly. In pure water if takes at least 6 hours for the matrix to swell and breakdown. More preferably this process of breakdown takes 24 to 48 hours after which period, a gentle disturbance will complete collapse of the structure.

It will be appreciated by those skilled in the art that the release rate in soil may vary to that in water. It will also be appreciated that a disturbance in soil would occur from earth forms, larvae of soil dwelling organisms, livestock or similar such disturbances. In practice, a combination of these factors causes gentle disturbances that collapse the matrix structure once combined with moisture and/or other environments.

It is to be noted that no additional drying of the composition of step (c) is required where a liquid bio-matrix is used.

In the case of a fast release mixture, the granules and/or pellets formed are bio-stable, in that until they come into contact with substantial amounts of water, they are both bio-stable and thermo-stable. However, once water is introduced the structure of the granules/pellet breaks down rapidly, within minutes, to release the biological material. This rapid breakdown optimally occurs in less than one minute.

In the case of fast release compositions, it has been found that the pellets of step (g) break down faster than composition formed in step (c) of the above process.

According to a further aspect of the present invention there is provided a method of preparing a slow release or delayed release composition substantially as described above wherein said composition is a thermo-stable gel and the composition is capable of storage of the biological materials for a period of time. On contact with substantial amounts of water, the composition slowly releases the biological material.

The liquid bio-degradable bio-matrix can be obtained in a number of ways or arrived at through a number of methods of preparation. An example of this is the use of acacia gum mixed with distilled water and agitated; and the further addition of concentrated biological material. This mix can be left to stand for up to three hours before being mixed with the preparation of step (b).

Other alternative liquid bio-matrixes which are also biodegradable and/or thermo-stable can be used. For example the liquid bio-matrix disclosed in specification of U.S. Pat. No. 5,292,507.

According to a further object of the present invention there is provided a method of producing a method of preparation of a release composition as described above wherein the biological material is cellular or a micro-organism. The concentration of such biological material at the end of step (g) is hereafter referred to as the “cell concentration”. Preferably, the cell concentration is in the range 10⁵ cells to 10¹² cells gel, more preferably in the range 10⁸ to 10¹² cells go, more preferably in the range of 10⁹ to 10¹⁰ cell g⁻¹. Preferably, the biological material may be present in step (a) in a broth, or on a growing medium.

According to another aspect of the present invention there is provided a method of preparing a slow-release or delayed release composition substantially as described above wherein the biological material includes: a pesticide; a viricide; a bacteriacide; a fungicide; and a combination thereof.

According to a further aspect of the present invention there is provided a method for producing a thermo-stable biodegradable medium for storage of biological materials wherein the biological material is a vaccine selected from: a live vaccine; an oral attenuated vaccine; an encapsulated mycobacterium vaccine; and a combination thereof. Examples of the vaccine include Bacille Calmette and Guerin (B.C.G.).

For the purposes of this specification, the term “storage” means a stability of better than LT₅₀ with respect to the cell concentration of the biological material. That is, more than 50% of the cells (if cells are the biological material) are viable at the end of the storage period; or more than 50% of the non-living material is viable at the end of the storage period. Advantageously, LT₅₀ may be achievable after two months, four to six months, or 12 to 18 months.

For the purposes of this specification, the term “thermo-stable” means a range of temperatures in which the combination of bio-polymer and biological material is stable. This temperature range is 4° C. to 40° C., and preferably between 5° C. to 30° C.

According to another aspect of the present invention there is provided a method of preparing a slow-release or delayed release composition substantially as described above wherein the biological material is a micro-organism. Said micro-organism may be selected from the group Serratia, Pseudomonas, Xanthomonas, Rhizobium. Most preferably the micro-organism is Serratia entomophila.

According to another aspect of the present invention there is provided a slow-release or delayed release composition incorporating a biodegradable thermo-stable bio-matrix with a biological material therein, said composition being formed by the above described method.

According to another aspect of the present invention there is provided a fast release composition, suitable for application to a substrate (as defined above), the composition being produced by steps (a) to (c), or steps (a) to (e) of the method as described above.

According to a further aspect of the present invention there is provided a method of producing a fast release composition as described above wherein the steps (f) and (g) occur a substantial time after the immediately preceding step. Optimally, the steps (f) and (g) can occur immediately before the composition is applied to a substrate.

According to a further aspect of the present invention there is provided a composition, in granular form, produced in accordance with steps (a) to (c) or steps (a) to (e) of the method of preparation as described above, characterised in that to the composition can be added one or more clays or/and other dry powders or/and sufficient water, and that the composition is capable of being formed into pellets of the type described above.

According to a further aspect of the present invention there is provided a release composition, produced in accordance steps (a) to (d) of the method of preparation as described above.

According to another aspect of the present invention there is provided a slow-release composition in the form of a dough incorporating a biodegradable thermo-stable bio-matrix and biological material, produced by the steps (a) to (e) of the above described method, wherein said dough can be passed through a pelletiser and dried.

According to a further aspect of the present invention there is provided a bulk dough release composition produced in accordance with steps (a) to (f) of the method of preparation as described above, and wherein said dough is processed through a pelletiser to form pellets.

According to a further aspect of the present invention there is provided pellets produced in accordance with steps (a) to (g) of the method described above.

Preferably, the pellets are of a size that can easily be drilled into soil or other substrate, either alone or in combination with seeds.

According to another aspect of the present invention there is provided a slow-release composition in the form of a powder, as prepared by steps (a) to (c) of the above described method, wherein said powder, when mixed with a least one clay and sufficient water, forms a dough capable of passage through a pelletiser.

According to another aspect of the present invention there is provided a slow-release composition in the form of a powder, as prepared by steps (a) to (d) of the above described method, wherein said powder, when mixed with sufficient water, forms a dough capable of passage through a pelletiser.

Optionally, the bio-matrix of step (a) will be between 0-50% water by weight. It will be appreciated that the powdered mix after step (c) of the method, or the dough formed after step (e) of the method, is also suitable for storage of the biological material until it is required to be applied to the substrate.

Optionally, the release composition can be used to stabilize biological material for transportation and/or storage and/or delivery.

According to a further aspect of the present invention there is provided the use of the release component for delivery of biological material via a spray for application to plants and/or animals, characterised in that the biological material includes an active ingredient to be sprayed over plants and/or animals.

According to another aspect of the present invention there is provided a spray solution which includes at least one release composition.

According to a still further aspect of the present invention there is provided a method of inoculating a plant seed with a biological material, said method including the steps of;

(a) selecting at least one biological material to be used as an inoculant;

(b) preparing the composition by the above described method;

(c) adding the composition to water and mixing to release the biological material into the solution;

(d) soaking the plant seed in said solution to allow the biological material to coat the plant seed.

According to a further aspect of the present invention there is provided a method of inoculating a plant seed with a biological material substantially as describe above, said method including, a further step after step (b), of (bi): adding a powdered compound to the composition, said powdered compound being selected from the group: a second biological material, a dried and powdered composition, a dried and powdered bio-polymer matrix containing a second or a third biological material, a chemical, and a combination of these. Preferably, the plant seed can be dried at room temperature before drilling or seed broadcast. Preferably, more than one inoculant may be used in step (a) above, each inoculant being for a different purpose. As the bio-matrix is thermo-stable and bio-stable, the seeds need not be drilled or sown immediately after the inoculation process.

According to a further aspect of the present invention there is provided seed inoculated by the method as described above.

According to a further aspect of the present invention there is provided a composition for application to a substrate (as hereinbefore defined) wherein said composition includes:

(a) one or more fast release compositions (substantially as hereinbefore described); and

(b) one or more slow release compositions.

### BEST MODES FOR CARRYNG OUT THE INVENTION

### EXAMPLE 1

A slow release gel is made with xanthan gum as the bio-polymer and Serratia entomophila as the biological material. The cell concentrations are set out in Table 1.

To 5 grams of dry xanthan gum is added 5 grams monounsaturated oil. The mixture is agitated at room temperature for between 5-10 minutes to form a suspension.

90 grams of the micro-organism concentrate is added to the suspension. The mix is agitated for a further 10 minutes at room temperature. The result is a gel matrix.

Equal portions of diatomaceous earth and talc are mixed to form 200 grams of powder. To this powder is added the 100 grams of gel. This can be done, for example, by drying and crumbling the blend of the gel and diatomaceous earth and talc, or by other known means.

To 300 grams of this mixed powder is added 27 grams of bentonite and up to 180 grams of distilled water. This mixture is homogenised and forms a dough. The mixture is passed through a pelletiser (of known type) and/or a die to form pellets of predetermined size and thickness. These pellets are then air dried to between 10-40% moisture content.

The results of six tests using the method of example 1 are shown in table 1 below. A comparison is shown of the survival of Serratia entomophila in broth at 20° C.

TABLE 1
Example 1

| | Initial | Survival - | Survival - | Survival - | Survival - | |
|---|---|---|---|---|---|---|
| | concentration | 1 month | 2 months | 4 months | 6 months | LT₅₀ |
| Sample # | cfu g⁻¹ | cfu g⁻¹ | cfu g⁻¹ | cfu g⁻¹ | cfu g⁻¹ | days |
| 60 | 6.08 × 10⁷ | 2.72 × 10⁹ | 1.64 × 10⁹ | 5.02 × 10⁸ | 1.52 × 10⁸ | 60-120 |
| 61 | 1.32 × 10⁹ | 6.98 × 10⁹ | 3.05 × 10⁹ | 1.23 × 10⁹ | 6.81 × 10⁸ | 120-180 |
| 62 | 2.77 × 10⁹ | 6.94 × 10⁹ | 7.53 × 10⁹ | 4.42 × 10⁶ | 6.68 × 10⁶ | 60-120 |
| 63 | 3.53 × 10⁹ | 4.59 × 10⁹ | 1.24 × 10⁹ | 4.98 × 10⁹ | 3.87 × 10⁹ | >180 |
| 64 | 8.25 × 10⁸ | 3.02 × 10⁹ | 1.75 × 10⁹ | 3.78 × 10⁹ | 1.78 × 10⁹ | >180 |
| 213 | 4.65 × 10⁹ | — | — | 3.72 × 10⁹ | 2.04 × 10⁹* | ˜165 |

Comparison

| | | | | | LT₅₀ |
|---|---|---|---|---|---|
| | Initial cfu g⁻¹ | 1 week cfu g⁻¹ | 2 week cfu g⁻¹ | 3 week cfu g⁻¹ | Days |
| Broth at 20° C. | 6.67 × 10¹⁰ | 4.52 × 10¹⁰ | 1.22 × 10¹⁰ | 3.83 × 10⁸ | <14 |

### EXAMPLE 2

A slow release gel is made with xanthan gum as the bio-polymer and Serratia entomophila as the biological material. The cell concentrations are set out in Table 2.

To 7.5 grams of dry xanthan gum is added 42.5 grams distilled water. The mixture is agitated at room temperature for between 5-10 minutes to form a suspension. Alternatively, a 50% solution of xanthan gum medium may be used for the gel.

50 grams of the micro-organism concentrate is added to the suspension. The mix is agitated for a further 10 minutes at room temperature. The result is a gel matrix.

Equal portions of diatomaceous earth and talc mixed to form 100 grams of powder. To this powder is added 100 grams of gel. This can be done, for example, by drying and crumbling the blend of the gel and diatomaceous earth and talc, or by other known means.

To 200 grams of this mixed powder is added 20 grams of bentonite and between 75-95 grams of distilled water. This mixture is homogenised and forms a dough. The mixture is passed through a pelletiser (of known type) and/or a die to form pellets of predetermined size and thickness. These pellets are then air dried to between 10-40% moisture content.

The pellets, depending on the die, may vary in size from 0.1-20 mm in diameter and from 0.1-20 mm in length. Preferably, if the pellets are to be drilled, a diameter of less than 3 mm is used. The pellets are stored in the absence of moisture at room temperature, until required.

Data for the survival of micro-organisms in a slow release pellet form over a period of months from 1-6 is shown in Table 2.

Alternatively, the powdered mix before the addition of the bentonite, or the dough after the addition of water, may be stored in the absence of moisture. The additional steps described above can be performed in a one or two stage process, depending on when the pellets or the composition is needed. Thus the next step can be separated in time from the immediately preceding mixing step and also from the step of addition of water. Alternatively, these steps can be performed immediately one after the other, and immediately before the pellets are to be used.

The pellets, if small, can be mixed with seed and passed through a drill in known manner and directly drilled into the soil. Alternatively, granules or pellets from the composition may be broadcast in known manner to a substrate of plants or soils or a combination thereof.

TABLE 2
Example 2

| | Initial | Survival - | Survival - | Survival - | Survival - 6 | |
|---|---|---|---|---|---|---|
| | Concentration | 1 month | 2 months | 4 months | months | LT₅₀ |
| Sample # | cfu g⁻¹ | cfu g⁻¹ | cfu g⁻¹ | cfu g⁻¹ | cfu g⁻¹ | days |
| 14 | 1.46 × 10¹⁰ | 2.63 × 10⁹ | 4.76 × 10⁹ | 2.76 × 10⁸ | 7.90 × 10⁵ | 60-120 |

### EXAMPLE 3

A slow release gel is made with guar gum as the bio-polymer and Serratia entomophila as the biological material. The cell concentrations are set out in Table 3.

To 5.6 grams of dry guar gum is added 0.5 grams of 0.5M sodium hydroxide. The mixture is agitated at room temperature for between 5-10 minutes to form a suspension.

100 grams of the micro-organism concentrate is added to the suspension. The mix is agitated for a further 10 minutes at room temperature. The result is a gel matrix.

Equal portions of diatomaceous earth and talc are mixed to form 150 grams of powder. To this powder is added 106.1 grams of gel. This can be done, for example, by drying and crumbling the blend of the gel and diatomaceous earth and talc, or by other known means.

To 256.1 grams of this mixed powder is added 18 g of bentonite and up to 70 grams of distilled water. This mixture is homogenised and forms a dough. The mixture is passed through a pelletiser (of known type) and/or a die to form pellets of predetermined size and thickness. These pellets are then air dried to between 10-40% moisture content.

A second sample is made by comparison without sodium hydroxide. The same method as above is used, however the quantities used are: 2.1 grams of dry guar gum added to 60 grams of micro-organism concentrate. The mix is agitated for 10 minutes at room temperature. The result is a gel matrix. A further 110-grams of diatomaceous earth-talc powder (mixed in equal proportions) is added to the gel matrix. After this, a further 10 grams of bentonite and 135 grams of distilled water is added. This mixture is formed into a dough and pelletised as above.

The results for both treatments are shown in Table 3 below.

TABLE 3
Example 3

| | Initial | Survival - | Survival - | Survival - | |
|---|---|---|---|---|---|
| | Concentration | 1 month | 2 months | 4 months | LT₅₀ |
| Sample # | cfu g⁻¹ | cfu g⁻¹ | cfu g⁻¹ | cfu g⁻¹ | days |
| 148 | 6.25 × 10⁹ | 8.83 × 10⁹ | 5.56 × 10⁹ | 1.45 × 10⁹ | 60-120 |
| 149 | 5.31 × 10⁹ | 8.58 × 10⁹ | 5.51 × 10⁹ | 2.64 × 10⁷ | 60-120 |

### EXAMPLE 4

The same composition and method was used as for Example 1, however the microbe Pseudomonas fluorescence was used in the microbial concentrate.

Results were taken of the cell concentration initially and after two months. The results are shown below in Table 4.

TABLE 4
Example 4

| | Initial | Survival- | |
|---|---|---|---|
| Sample | Concentration | 2 month | LT₅₀ |
| # | cfu g⁻¹ | cfu g⁻¹ | days |
| 288 | 4.18 × 10⁹ | 1.26 × 10¹⁰ | >60 |

### EXAMPLE 5

A fast release composition is made by combining 20 grams of acacia gum with 30 grams of distilled water. The mix is agitated at room temperature. To this mix is added 50 grams of concentrated biological material containing Bacillus mycoides. This mixture is agitated at room temperature and left to stand for between 1½ to 3 hours at room temperature.

A powder is formed of diatomaceous earth and talc in the ratio 50:50 (by weight). The solution of acacia gum and biological material is added to 200 grams of the powdered mix and mixed thoroughly.

A second mixture was prepared using the above method. However, the quantities used were 16 grams of acacia gum, 8 grams of 0.5% yeast, 12 grams of distilled water, 36 grams of Trichoderma spores and 130 grams of a 50:50 mix of diatomaceous earth and talc (by weight).

Either mixture can either be stored at room temperature until required.

The survival rates for the biological materials of Example 5 were tested and the results are set out in Table 5 below.

TABLE 5
Example 5

| | | Initial | 3 months | 6 months | 8 months | LT₅₀ |
|---|---|---|---|---|---|---|
| Sample # | Organism | cfu g⁻¹ | cfu g⁻¹ | cfu g⁻¹ | cfu g⁻¹ | days |
| 186 | Bacillus Mycoides | 8.47 × 10⁵ | 8.25 × 10⁵ | 8.00 × 10⁵ | 4.3 × 10⁵ | >240 |
| 171 | Trichoderma | 2.96 × 10⁹ | 2.02 × 10⁹ | 1.81 × 10⁹ | 7.81 × 10⁸ | 120-180 |

### EXAMPLE 6

A fast release composition was made by combining 2.5 grams of gellan gum with 50 grams of concentrated biological material containing Bacillus mycoides. This mixture is agitated at room temperature and left to stand for between 1½ to 3 hours at room temperature.

A powder is formed of diatomaceous earth and talc in the ratio 60:40 (by weight). The solution of gellan gum and biological material is added to 100 grams of the powdered mix and mixed thoroughly. To this resulting mixture a further 15 grams of bentonite and 100 grams of distilled water is added.

A second mixture was prepared using the same composition and method as used above, however the microbe Pseudomonas fluorescence was used in the microbial concentrate.

A third mixture was prepared using the above method, however the quantities used were 3 grams of gellan gum, 9 grams of 0.5% yeast, 18-grams of distilled water, 28 grams of Tricitoderna spores and 120 grams of a 50:50 mix of diatomaceous earth and talc (by weight), 15 grams of bentonite and 110 grams of distilled water.

Results were taken of the cell concentration initially and after various monthly intervals thereafter. The results are shown below in Table 6.

TABLE 6
Example 6

| | | Initial | 2 months | 3 months | 6 months | 8 months | LT₅₀ |
|---|---|---|---|---|---|---|---|
| Sample # | Organism | cfu g⁻¹ | cfu g⁻¹ | cfu g⁻¹ | cfu g⁻¹ | cfu g⁻¹ | days |
| 185 | Bacillus | 1.61 × 10⁶ | — | 1.40 × 10⁶ | 1.02 × 10⁶ | 8.03 × 10⁵ | >240 |
| | mycoides | | | | | | |
| 289 | Pseudomonas | 1.73 × 10⁹ | 1.86 × 10⁹ | — | — | — | >60 |
| | fluoresence | | | | | | |
| 172 | Trichoderma | 1.2 × 10⁷ | — | 1.3 × 10⁷ | 4.6 × 10⁶ | 6.7 × 10⁵ | 90-180 |

### EXAMPLE 7

A fast release composition is made by combining 3.5 grams of starch with 50 grams of hot water (70° C. to 80° C.). The mix is held in this state for 10 minutes and then cooled to room temperature. To this mix is added 50 grams of concentrated biological material (in this example Serratia eittomophila). This mixture is agitated at room temperature and left to stand for between 1½ to 3 hours at room temperature.

A powder is formed of diatomaceous earth and talc in the ratio 60:40 (by weight). The solution of starch and biological material is added to 175 grams of the powdered mix, 26 grams of bentonite and 150 grams of distilled water, and the resulting solution is mixed thoroughly.

A similar fast release composition is made by combining 6 grams of starch with 100 grams of concentrated biological material (Serratia entomophila). A powder is formed of diatomaceous earth and talc in the ratio 60:40 (by weight). The solution of starch and biological material is added to 175 grams of the powdered mix, 12 grams of bentonite and 180 grams of distilled water and the resulting solution is mixed thoroughly.

The survival rates for both of the above options were tested at 2½ months, 3½ months and 5½ months. The results are set out in Table 7 below.

TABLE 7
Example 7

| Sam- | Initial | Survival - | Survival - | Survival - | |
|---|---|---|---|---|---|
| ple | Concentration | 2½ months | 3½ months | 5½ months | LT₅₀ |
| # | cfu g⁻¹ | cfu g⁻¹ | cfu g⁻¹ | cfu g⁻¹ | days |
| 208 | 1.79 × 10⁹ | 1.14 × 10⁹ | 2.8 × 10⁹ | 8.59 × 10⁸ | >165 |
| 225 | 4.77 × 10⁹ | 5.26 × 10⁹ | 2.49 × 10⁹ | 1.25 × 10⁹ | ˜165 |

### EXAMPLE 8

The success or otherwise of the biomatrix (biofungicide) in aiding root rot disease control was compared against a control sample where no treatment occurred and a seed treated with a known chemical fungicide.

The seeds chosen were pea seeds which are prone to Aphanontyces root rot. The microbe Bacillus mycoides is known to control this disease, as is the chemical fungicide used in the trial as a positive control. It was expected that the positive control fungicide would have a heightened effect as the fungicide has a broad range of fungicidal properties.

A matrix treated according to Example 6 was placed at the bottom of each drilled hole, upon which the pea seed was placed.

The results of the test are shown below in Table 8 where the better yield of the plant relates to the lower presence of Aphanomyces root rot.

TABLE 8
Example 8

| | | | Pods | Pod | Yield of |
|---|---|---|---|---|---|
| | Initial | % Plot | per | Weight per | Pods per |
| Treatment | Spore g⁻¹ | Stand | plant | plant (g) | Plot (g) |
| Chemical | 870 | 92.78 | 2.78 | 15.07 | 503.15 |
| fungicide | | | | | |
| Control | 4.0 × 10¹ | 77.22 | 2.59 | 14.37 | 399.62 |
| Biofungicide | 1.5 × 10⁶ | 79.44 | 3.26 | 16.86 | 482.20 |

It will be appreciated by those skilled in the art that a number of compounds, biological material or other chemicals can be added to the suspension prior to the inoculation of the plant seed. Such compounds can be for the promotion of plant growth, the promotion of growth of the animal feeding off the plant, etc.

It will also be appreciated that the suspension generated in the Example 8 above may be sprayed in known manner, to broadcast the biological material onto a substrate. Such spraying can be done either before or after planting.

It will also be further appreciated that the seeds in Example 8 above may be inoculated with the bio-matrix by soaking the seeds in the suspension prior to drilling.

### EXAMPLE 9

A further experiment to assess the success or otherwise of the bio-matrix in control of grass grubs at filed level was compared against a control sample where no treatment occurred and a field drilled with two separate release formulations containing Serratia entornyphila. Serratia entoinyphila is a microbe that is efficient at controlling grass grub, a common disease in pasture grass.

The above samples were compared to positive controls where a liquid, product was applied through a modified see drill onto the paddock.

The first release solution is that produced by example 2 and the second, by example 7.

The paddock kept isolated via a fence. Presence of the disease in grass grubs was tested after 6 weeks in the case of the fast release formulation and after 10 weeks in the case of the slow release formulation. The results are shown below in Table 9. Ideally the higher the percentage of disease prevalence in the grass grub, the better the rate of success of the formulation i.e. the higher the occurrence of the disease in the grass grub relates to the presence of Serratia.

TABLE 9
Example 9

| | Sample Set 1 | Sample Set 2 | Trial |
|---|---|---|---|
| Treatment | Average | Average | Average |
| Control | 9.2% | 2.7% | 6.0% |
| Drilled Fast Release | 25.2% | 5.0% | 15.1% |
| Drilled Slow Release | 24.7% | 18.4% | 21.6% |
| Liquid (into SR) | 23.5% | 32.3% | 27.9% |
| Liquid (into FR) | 25.7% | 9.1% | 17.4% |

Aspects of the present invention have been described by way of example only and it should be appreciated that modifications and additions may be made thereto without departing from the scope thereof.

The claims defining the invention are:

1. A method of preparation of a release composition, which method comprises the steps of:
(a) preparing a bio-degradable bio-matrix either as a gel or a liquid, incorporating a biological material;
(b) preparing a dry powder of one or more inert compounds selected from the group including: talc; bentonite; diatomaccous earth; and combinations thereof; and
(c) mixing the preparation of step (a) and the preparation of step (b) to form a homogenous mix;
and wherein the bio-matrix is selected from the group: xanthan gum; acacia gum; guar gum; gellan; starch; and a combination thereof;
and wherein the biological material is selected from: a bio-inoculant, a micro-organism, biological cells, a part or parts of a biological cell, a vaccine, at least one pharmaceutical compound, at least one enzyme, at least one hormone, at least one protein; at least one biochemical, at least one biologically unstable composition; and a combination thereof;
and wherein said composition is thermo-stable and bio-stable when stored with no more than trace amounts of water.

2. A method of preparation of a release composition as claimed in claim 1 wherein said biological material is combined with a chemical composition.

3. A method as claimed in claim 1 wherein said organic material is selected from: corn cobs; chaff; straw; or a combination thereof.

4. A method of preparation of a release composition as claimed in any one of the previous claims wherein said method includes a further step, after step (c), as follows:
(d) mixing the composition formed in step (c) with at least one further inert compound selected from the group including: talc; bentonite; diatomaceous earth; and combinations thereof.

5. A method of preparation of a release composition as claimed in claim 4 wherein the inert compound in step (d) (if present) is powdered bentonite.

6. A method of preparation of a release composition as claimed in any one of the previous claims wherein said method includes a further step, after step (c), and after step (d) if present, as follows:
(e) repeating steps (a) to (c) at least once more with a different bio-matrix and/or a different biological material; and mixing together the homogenous mix resultant from all steps (c).

7. A method of preparation of a release composition as claimed in claim 6 wherein said method includes a second step (d) after step (c), and wherein the composition is a fast release composition (as hereinbefore defined).

8. A method of preparation of a release composition as claimed in claims 4 to 7 wherein said method includes a further step after step (c) and after step (d) if present, as follows:
(f) mixing the resultant mix of any step (d) with water, to form a dough.

9. A method of preparation of a release composition as claimed in claim 8, wherein said method includes further steps after step (f) as follows:
(g) forcing through or extruding the dough of step (f) to form pellets.

10. A method as claimed in claim 9 wherein the pellets resulting from step (h) have a size of 0.1-20 mm diameter and up to 30 mm long and are of a shape selected from round, ovoid, cylindrical, spherical and rectangular.

11. A method as claimed in claim 10 wherein the pellets resulting from step (g) are in the range 0.1 mm to 10 mm in diameter and up to 10 mm long.

12. A method of producing a release composition as claimed in any one of the previous claims wherein the biological material is cellular or is micro-organism.

13. A method as claimed in claims 9 to 12 wherein the concentration of biological material, at the end of step (g), is in the range 10⁵ cells to 10¹² cells g⁻¹.

14. A method as claimed in claim 13 wherein the cell concentration is in the range 10⁸ to 10¹² cells g⁻¹.

15. A method as claimed in claim 13 wherein the cell concentration is in the range 10⁹ to 10¹⁰ cells g⁻¹.

16. A method as claimed in any one of the previous claims wherein the biological material in step (a) is in a state selected from: a broth or a growing medium or a combination thereof.

17. A method of preparing a release composition as claimed in any one of the previous claims wherein the biological material is selected from: a pesticide; a viricide; a bacteriacide; a fungicide; and a combination thereof.

18. A method of preparation of a release composition as claimed in any one of the previous claims wherein the biological material is a vaccine selected from: a live vaccine; an oral attenuated vaccine; an encapsulated mycobacterium vaccine; and a combination thereof.

19. A method as claimed in claim 18 wherein the vaccine Bacille Calmette and Guerin (B.C.G.) is used.

20. A method as claimed in any one of the previous claims wherein the biological material is cellular or a micro-organism and storage of the composition is at a stability of better than LT₅₀ with respect to the cell concentration for the duration of the storage period.

21. A method as claimed in claim 20 wherein the temperature range for storage is 4° C. to 40° C.

22. A method as claimed in either claim 20 or claim 21 wherein the temperature range for storage is 5° C. to 30° C.

23. A method of preparation of a composition as claimed in any one of claims 9 to 22, wherein said method includes further step (h) after step (g) of:
(h) drying the pellets or composition of steps (c) to (e) at room temperature characterised in that said composition is a slow release composition (as herein before defined).

24. A method of preparation of a slow release composition as claimed in claim 23 wherein said bio-degradable bio-matrix has between 15 to 40% water by weight at the end of step (g).

25. A method of preparing a slow-release composition as claimed in claim 23 or 24 wherein the biological material is a micro-organism.

26. A method of preparing a slow-release composition as claimed in claim 25 wherein said micro-organism may be selected from the group Serratia, Pseudomonas, Xanthomonas, Rhizobium, and a combination thereof.

27. A method of preparing a slow-release composition as claimed in claim 25 wherein the micro-organism is Serratia entomophila.

28. A method of preparation of a composition as claimed in any one of claims 1 to 22 wherein the compositions formed are both bio-stable and thermo-stable and wherein when introduced to water, in less than 10 minutes, the physical structure of the compositions disintegrates to release the biological material, characterised in that said composition is a fast release composition (as herein before defined).

29. A method of preparing a fast release compositing as claimed in claim 28 wherein the time is less than one minute.

30. A method of producing a fast release composition as claimed in either claims 28 or 29 wherein the steps (f) and (g) occur at any time after the preceding step and before the LT₅₀ time limit.

31. A method of producing a fast release composition as claimed in claim 30 wherein, the steps (f) and (g) occur immediately before the composition is applied to a substrate.

32. A release composition formed by the method of anyone of the previous claims.

33. A release composition produced by the method of any one of claims 1 to 31 where the composition is used to stabilize biological material for any one of: transportation; storage; delivery and a combination thereof.

34. A slow release composition formed by any one of claims 23 to 27.

35. A fast release composition formed by any one of claims 7 and 28 to 31.

36. A dough produced by the method as claimed in claim 8.

37. Pellets produced by the method as claimed in any one of claims 9 to 11.

38. A spray solution that includes at least one release composition as claimed in claims 32 to 35.

39. The use of a spray composition as claimed in claim 38 for delivery of biological material for application to plants and animals, characterised in that the biological material includes an active ingredient to bc sprayed over plants and animals.

40. A method of inoculating a plant seed with a biological material, said method including the steps of;
(a) selecting at least one biological material to be used as an inoculant;
(b) preparing the composition by the method as claimed in any one of claims 1 to 31;
(c) adding the composition to water and mixing to release the biological material into the solution; and
(d) soaking the plant seed in said solution to allow the biological material to coat the plant seed.

41. A method of inoculating a plant seed with a biological material as claimed in claim 40 wherein said method includes a farther step after step (b), of:
(bi) adding a further powdered compound to said composition, said powdered compound being selected from the group: a second biological material, a dried and powdered composition, a dried and powdered bio-polymer matrix containing at least a second biological material, a chemical, and a combination of these.

42. A method as claimed in either claim 40 or claim 41 wherein more than one inoculant is used in step (a) each inoculation being for a different purpose.

43. A method as claimed in either claim 41 or claim 42 wherein the plant seed is dried at room temperature after inoculation, but before drilling or seed broadcast.

44. Seed inoculated by the method as claimed in any one of claims 40 to 43.

45. A composition for application to a substrate (as hereinbefore defined) wherein said composition includes:
(a) one or more fast release compositions as produced by the method of any one of claims 7 and 28 to 31; and
(b) one or more slow release compositions as produced by the method of any one of claims 23 to 27.
